(19) United States
(12) Patent Application Publication
(10) Pub. No.: US 2023/0147942 A1
(43) Pub. Date: May 11, 2023

(54) PHARMACEUTICAL COMPOSITION COMPRISING NEW LACTOBACILLUS PLANTARUM KC3 STRAIN AND LEONURUS JAPONICUS EXTRACT AS ACTIVE INGREDIENTS FOR PREVENTING OR TREATING RESPIRATORY DISEASE AND USE THEREOF

(71) Applicants: KT&G CORPORATION, Daejeon (KR); CKD BIO CORPORATION, Seoul (KR)

(72) Inventors: Han Jae SHIN, Daejeon (KR); Young Chul LEE, Daejeon (KR); Do Hoon KIM, Daejeon (KR); In Ock LEE, Incheon (KR); Min Ho SEO, Gwangmyeong-si (KR); Bo Hye HEO, Ansan-si (KR); Yoo Il KIM, Seo-gu (KR); Woo Ri KIM, Incheon (KR); Yoo Jin KWON, Ansan-si (KR); Byoung Kook KIM, Seoul (KR); In Suk CHOI, Seoul (KR)

(73) Assignees: KT&G CORPORATION, Daejeon (KR); CKD BIO CORPORATION, Seoul (KR)

(21) Appl. No.: 17/915,306

(22) PCT Filed: Aug. 24, 2021
(86) PCT No.: PCT/KR2021/011257

§ 371 Date: Sep. 28, 2022

(30) Foreign Application Priority Data
Aug. 31, 2020 (KR) 10-2020-0110265

(51) Int. Cl. A61K 35/747; A61K 36/533; A61P 11/00; A61P 29/00; A61K 9/14; A61K 9/20; A61K 9/48; A61K 9/00; A61K 47/26; C12N 1/20; A23L 33/135; A23L 33/105; A23L 2/52

(52) U.S. Cl. CPC A61K 35/747; A61K 36/533; A61P 11/00; A61P 29/00; A61K 9/145; A61K 9/143; A61K 9/2059; A61K 9/2018; A61K 9/2013; A61K 9/4866; A61K 9/4858; A61K 9/0095; A61K 9/0019; A61K 47/26; C12N 1/205; A23L 33/135; A23L 33/105; A23L 2/52; C12R 2001/25

(57) ABSTRACT

Disclosed are a pharmaceutical composition for preventing or treating a respiratory disease, and health functional food, a food composition, and a quasi-drug composition for preventing and ameliorating a respiratory disease, each including, as active ingredients, a Lactobacillus plantarum KC3 strain and a Leonurus japonicus extract. The composition including the Lactobacillus plantarum KC3 strain (Accession No: KCTC13375BP) and the Leonurus japonicus extract as active ingredients according to an aspect has a defense effect against respiratory damage caused by air pollutants such as fine dust and can inhibit expression of IL-17A, TNF-α, and CXCL-1, thereby being able to effectively treat or prevent a respiratory disease including chronic obstructive pulmonary disease (COPD). In addition, the effect of the active ingredient combination above results in a synergistic inhibition or treatment effect on bronchial inflammation by the administration compared to the existing therapeutic effect of a respiratory inflammatory disease of each of the Leonurus japonicus extract and the lactic acid bacteria KC3. Thus, the present disclosure can be usefully utilized for the prevention or treatment of a respiratory disease.

### TECHNICAL FIELD

The present disclosure relates to a pharmaceutical composition for preventing or treating a respiratory disease, and a health functional food, a food composition, and a quasi-drug composition for preventing or ameliorating a respiratory disease, each including, as active ingredients, a Lactobacillus plantarum KC3 strain and a Leonurus japonicus extract.

### BACKGROUND ART

Generally, an inflammatory response is a defensive response process of the living body associated with restoration and regeneration of a damage part, when an invasion causing some organic changes in the cells or tissues of the living body is applied. Thus, spots for a series of such reactions include local blood vessels, various tissue cells in body fluids, immune-related cells, and the like. With recent developments in molecular biology, attempts have been made to understand the involvement of cytokines in inflammatory diseases at a molecular level, and factors affecting such diseases have been investigated one by one.

Accordingly, due to the production and actions of cytokines, such as IL-4, IL-5, IL-13, and the like, and immunoglobulin E that are involved in the activation of inflammatory cells, biosynthesis of cysteine leukotrienes including eosinophils secreted from inflammatory cells may be the main causes of inflammation, allergic reactions, and asthma caused by inflammation and allergic reactions. Thus, a number of studies have been conducted to develop drugs to inhibit the production of cytokines and immunoglobulin E.

In addition, chronic obstructive pulmonary disease (COPD) should be appropriately treated, distinguished from asthma that is characterized mainly by reversible airflow obstruction and allergic bronchial inflammatory responses. However, current COPD treatments only provide symptomatic alleviation, and none of recent treatments has demonstrated fundamental therapeutic effects of COPD as a clinical result.

Therefore, for use as a therapeutic agent using antibodies to various types of cytokines and chemokines that are typically associated with a respiratory disease, there is a demand for the development of therapeutic agents using various resources, particularly extracts and Lactobacillus strains, which are natural products whose safety is already known.

### DESCRIPTION OF EMBODIMENTS

### Technical Problem

An aspect provides a pharmaceutical composition for preventing or treating a respiratory disease, the pharmaceutical composition including, as active ingredients, a Lactobacillus plantarum KC3 strain and a Leonurus japonicus extract.

Another aspect provides health functional food, a food composition, and a quasi-drug composition for preventing or ameliorating a respiratory disease, each including the pharmaceutical composition that includes, as active ingredients, the Lactobacillus plantarum KC3 strain and the Leonurus japonicus extract, for preventing or treating a respiratory disease.

Another aspect provides a method of preventing or treating a respiratory disease, the method including administering the composition including, as active ingredients, the Lactobacillus plantarum KC3 strain and the Leonurus japonicus extract, to a subject in need thereof.

Other purposes and advantages of the present disclosure will become more obvious with the following detailed description, claims, and drawing. Contents not described herein will be sufficiently recognized and inferred by those skilled in the technical field of the present application or in a similar technical field therewith, and thus descriptions of such contents will be omitted.

### Solution to Problem

An aspect provides a pharmaceutical composition for preventing or treating a respiratory disease, the pharmaceutical composition including, as active ingredients, one or more selected from the group consisting of a Lactobacillus plantarum KC3 strain (Accession No: KCTC13375BP), a culture thereof, a fermentation solution thereof, a lysis solution thereof, an extract thereof, and a concentrate of the culture and a Leonurus japonicus extract.

The Lactobacillus plantarum KC3 strain (Accession No: KCTC13375BP, also referred to as “CKDB-KC3”) as defined herein refers to, as a new strain isolated from kimchi, a strain that does not produce biogenic amines from one or more amino acid precursors selected from the group consisting of tyrosine, histidine, ornithine and lysine.

The Lactobacillus plantarum KC3 strain as defined herein includes a nucleic sequence of SEQ ID NO: 1 in 16s rRNA.

The culture may be a culture itself obtained by culturing the Lactobacillus plantarum KC3 strain, or a culture supernatant obtained by removing the strain therefrom, a concentrate of the culture, or a freeze-dried product of the culture.

The lysis solution may refer to a product obtained by fragmentizing the strain itself by applying a chemical or physical force thereto.

The culture may refer to, regardless of the form of a culture, a material including some of or all of materials contained in a medium where the strain is cultured. For example, the culture may refer to a material including a metabolite or a secretion resulting from culturing the strain, or a lysate of the material, and the strain itself may also be contained in the culture. In addition, the culture may refer to inclusion of a fermented product.

The one or more selected from the group consisting of the Lactobacillus plantarum KC3 strain, the culture thereof, the fermentation solution thereof, the lysis solution thereof, the extract thereof, and the concentration of the culture may be included in an amount, based on the total weight of the composition, in a range of about 1 wt % to about 99.99 wt %, for example, about 1.5 wt % to about 99.99 wt % or about 2 wt % to about 99.99 wt %.

The Lactobacillus plantarum KC3 strain of an aspect may be obtained as follows. For example, although not limited thereto, processes of isolating and identifying a microorganism for the Lactobacillus plantarum KC3 strain of the present invention may be obtained as follows.

The new Lactobacillus plantarum KC3 strain of the present disclosure (“CKDB-KC3”) can be obtained from kimchi of different types from different regions in South Korea. Preferably, the new strain can be obtained from kimchi from commercially available products such as Jonggajip® and CJ Bibigo®, or from home-made Korean kimchi prepared in restaurants, homes, and temples in North Gyeongsang Province, North Chungcheong Province, Gyeonggi Province, North Jeolla Province, South Jeolla Province, and the like. More preferably, the new strain can be obtained from kimchi from Jeonju city in North Jeolla Province. Even more preferably, although not limited thereto, the new strain can be obtained from kimchi prepared by the following six steps: Step 1: raw materials for kimchi seasoning are prepared by six stages (a) to (f): (a) (first stage) a cabbage from North Jeolla Province is prepared and cut into two pieces after getting rid of inedible portions, and then, in a container for salting, salt in an amount in a range of, based on the weight of the cabbage, about ⅕ part by weight (w/w) to about 1/30 part by weight (w/w), preferably, about 1/10 part by weight (w/w) to about 1/120 part by weight (w/w), is dissolved in water, and the divided cabbage pieces are soaked in the salted water, and after being taken out of the salted water, salt was sprinkled on layers between the cabbage leaves, and the cabbages are salted for about 3 hours to about 8 hours, preferably, about 5 hours to about 6 hours, washed 2 times to 12 times, preferably, 3 times to 5 times, and placed on a large colander for dehydration; (b) (second stage) a Korean radish in an amount in a range of, based on the weight of the Korean cabbages, about ½ part by weight (w/w) to about 1/10 part by weight (w/w), preferably, about 1/3 part by weight (w/w) to about 1/6 part by weight (w/w), is prepared, trimmed and washed after getting rid of radish leaves, and cut into thin strips in 3 cm to 6 cm long, and then, great green onions, chives, and mustard leaves each in an amount in a range of, based on the weight of the Korean cabbages, about ⅕ part by weight (w/w) to about 1/30 part by weight (w/w), preferably, about 1/10 part by weight (w/w) to about 1/120 part by weight (w/w) are also trimmed and washed, and cut to the similar length with the Korean radish strips; (c) (third stage) garlic in an amount in a range of, based on the weight of the Korean cabbages, about 1/50 part by weight (w/w) to about 1/300 part by weight (w/w) preferably, about 1/80 part by weight (w/w) to about 1/120 part by weight (w/w), ginger in an amount in a range of, based on the weight of the Korean cabbages, about 1/100 part by weight (w/w) to 1/1000part by weight (w/w), preferably, about 1/300 part by weight (w/w) to 1/600 part by weight (w/w), and salted shrimp in an amount in a range of, based on the weight of the Korean cabbages, about 1/10 part by weight (w/w) to about 1/100 part by weight (w/w), preferably, about 1/120 part by weight (w/w) to about 1/40 part by weight (w/w) are finely minced, and anchovy sauce in an amount in a range of, based on the weight of the Korean cabbages, about 1/10 part by weight (w/w) to about 1/100 part by weight (w/w), preferably, about 1/120 part by weight (w/w) to about 1/40 part by weight (w/w) is prepared; (d) (fourth step) glutinous rice in an amount in a range of, based on the weight of the Korean cabbages, about 1/10 part by weight (w/w) to about 1/200 part by weight (w/w), preferably, about 1/120 part by weight (w/w) to about 1/80 part by weight (w/w) is soaked in water, and rice porridge is cooked using the same, and after cooling the rice porridge, the salted shrimp, anchovy sauce, garlic, and ginger prepared in the third stage are added thereto with red pepper powder in an amount in a range of, based on the weight of the Korean cabbages, about ½ part by weight (w/w) to about 1/40 part by weight (w/w), preferably, about ⅕ part by weight (w/w) to about 1/10 part by weight (w/w), and then, all the seasonings are mixed evenly; (e) (fifth step) after putting and mixing all the Korean radish strips, great green onion, chives, and mustard leaves that are all cut to the similar length as in the second stage, kimchi seasoning is made by seasoning with salt in an amount in a range of, based on the weight of the Korean cabbages, about ½ part by weight (w/w) to about 1/40 part by weight (w/w), preferably, about ⅕ part by weight (w/w) to about 1/10 part by weight (w/w), and sugar in an amount in a range of, based on the weight of the Korean cabbages, about ½ part by weight (w/w) to about 1/40 part by weight (w/w), preferably, about ⅕ part by weight (w/w) to about 1/10 part by weight (w/w); and (f) (sixth step) after spreading the kimchi seasoning evenly between the Korean cabbage leaves, each of the Korean cabbages is rolled by the outermost leaf and placed one by one in a container in a way that the cross section of the Korean cabbage faces up, and then, the container is stored in a low-temperature storage maintaining a temperature thereof between about 10° C. to about −10° C., preferably, between about 0° C. to about −2° C., so as to ripen the kimchi for about 3 months to about 5 years, preferably, about 6 months to about 2 years, thereby preparing the Korean cabbage kimchi as a raw material; Step 2: the raw materials for kimchi seasoning are inoculated onto an MRS medium, preferably, a modified MRS medium to which bromcresol purple and sodium azide are diluted with a peptone diluent and added, at a certain amount by a streak-plate method, thereby obtaining a medium inoculated with a strain;

Step 3: the strain-inoculated medium of Step 2 is cultured at a temperature in a range of about 27° C. to about 47° C., preferably, about 32° C. to about 39° C., for about 12 hours to about 72 hours, preferably, about 26 hours to about 52 hours, more preferably, about 33 hours to about 46 hours;

Step 4: the colonies of Step 3 are isolated purely from the MRS medium, preferable, the modified MRS medium to which bromcresol purple and sodium azide are diluted with a peptone diluent and added, thereby obtaining colonies that run yellow;

Step 5: the colonies of Step 4 are selected as tentative lactic acid bacteria; and

Step 6: the tentative strain selected in Step 5 is smeared on an MRS medium preferably an electrically-modified MRS medium, and cultured in an aerobic condition, thereby purely isolating a new Lactobacillus plantarum KC3 strain of the present disclosure having the following characteristics below.

As a result of identifying the strain purely isolated by the manufacturing process above, it is confirmed that the strain is a Gram-positive bacillus, grows well regardless of the presence of oxygen, and is negative for catalase and motility. The strain is also found not to grow at a temperature between 15° C. and 45° C., and based on that no gas from glucose and no ammonia from alginine are produced, the strain is confirmed to belong to the genus Lactobacillus.

In addition, the nucleic sequence (SEQ ID NO: 1), which is obtained by collecting the colonies grown in the MRS medium and performing double-stranded DNA sequencing (Solgent, Korea) thereon, is searched by BLAST to identify the strain. As a result, the strain shows a homology of 99% to the Lactobacillus plantarum, confirming that the new microorganism of the present disclosure is the strain belonging to the Lactobacillus plantarum species.

The new Lactobacillus plantarum KC3 (hereinafter referred to as “CKDB-KC3”) of the present disclosure is characterized by the following characteristics:

(1) Form of bacteria: Form of bacteria when cultured in MRS agar plate medium at 37° C. for 48 hours

\textcircled{1} Cell type: Bacillus

\textcircled{2} Mobility: None

\textcircled{3} Spore-forming ability: None

\textcircled{4} Gram staining: Positive

(2) Form of colony: Form of colonies when cultured in MRS agar plate medium at 37° C. for 48 hours

\textcircled{1} Shape: Round

\textcircled{2} Bulge: Convex

\textcircled{3} Surface: Smooth

\textcircled{4} Color: Milky-white

(3) Physiological properties

\textcircled{1} Temperature for growth and development

Temperature enable growth and development: 15° C. to 40° C.

Optimal temperature for growth and development: 36° C. to 38° C.

\textcircled{2} pH for growth and development

pH enable growth and development: 4.6 to 7.5

Optimal pH: 6.0 to 7.0

\textcircled{3} Effect on oxygen: Facultative anaerobic

(4) Catalase: Negative

(5) Gas generation: Negative

(6) Indole production: Negative

(7) Lactic acid production: Positive

(8) Biogenic amine production: Negative

Based on the results of identifying the microorganism and the bacteria characteristics above, the new strain isolated from kimchi was newly named Lactobacillus plantarum KC3 (also referred to as “CKDB-KC3”). Also, as described in the existing KR 10-2011883B, the Lactobacillus plantarum KC3 was deposited at the Korea Research Institute of Bioscience and Biotechnology (Accession No: KCTC13375BP) on October 20, 2017.

In addition, processes for a culture in the art, a concentrate of the culture, a dried material of the culture may be additionally performed (KR 10-1605516B disclosing “Method for Increasing Viability, Storage Stability, Acid Tolerance or Oxgall Tolerance of Lactic Acid Bacteria”).

The Leonurus japonicus extract as defined herein may be extracted by using a hydrophilic solvent. The hydrophilic solvent may include water, alcohol, C₁ to C₁₀ alcohol, or any mixture thereof. The alcohol may be, for example, a C₁ to C₁₀ compound, a C₁ to C₆ group, or a C₁ to C₄ group, each including one or more -OH groups. The alcohol may be, for example, methanol, ethanol, n-propanol, isopropanol, n-butanol, sec-butanol, isobutanol, tert-butanol, or a mixture thereof. In an embodiment, the extraction solvent used in the extraction of the Leonurus japonicus extract may be water, C₁ to C₄ alcohol, or any mixture thereof.

The one or more selected from the group consisting of the Lactobacillus plantarum KC3 strain(Accession No: KCTC13375BP), the culture thereof , the fermentation solution thereof, the lysis solution thereof, the extract thereof, and the concentrate of the culture and the Leonurus japonicus extract may be included as the active ingredients in the composition may be included at a relative mixing weight ratio by dry weight in a range of, for example, about 1 : 0.01 to about 100 (w/w), 1 : 0.5 to 50 (w/w), 1 : 0.1 to 10 (w/w), 1 : 0.3 to 5 (w/w), 1 : 0.5 to 3 (w/w), 1 : 0.5 to 1.5 (w/w), or 1 : 1 (w/w).

The Lactobacillus plantarum KC3 strain included as the active ingredient in the composition may be included at a concentration in a range of, for example, about 0.1×10⁹ CFU/cell to about 1.0×10⁹ CFU/cell, about 0.2×10⁹ CFU/cell to about 1.0×10⁹ CFU/cell, about 0.25×10⁹ CFU/cell to about 1.0×10⁹ CFU/cell, about 0.4×10⁹ CFU/cell to about 1.0×10⁹ CFU/cell, about 0.5×10⁹ CFU/cell to about 1.0×10⁹ CFU/cell, about 0.25×10⁹ CFU/cell to about 1.0×10⁹ CFU/cell, about 0.3×10⁹ CFU/cell to about 1.0×10⁹ CFU/cell, about 0.3×10⁹ CFU/cell to about 0.8×10⁹ CFU/cell, about 0.4×10⁹ CFU/cell to about 0.7×10⁹ CFU/cell, about 0.4×10⁹ CFU/cell to about 0.6×10⁹ CFU/cell, about 0.45×10⁹ CFU/cell to about 0.55×10⁹ CFU/cell. Accordingly, the one or more selected from the group consisting of the culture, the fermentation solution, the lysis solution, and the extract of the Lactobacillus plantarum KC3 strain, and the concentrate of the culture included as the active ingredients in the composition may be included to have the number of cells described above, and may be used after being diluted to have the number of cells. described above.

In detail, the Leonurus japonicus extract may include, for example, an extract soluble in: water or 10% to 100% (v/v) ethanol, or a mixture of alcohols; water or 10% to 45% (v/v) ethanol, or a mixture of alcohols; 20% to 40% (v/v) ethanol; 25% to 35% (v/v) ethanol; or 30% (v/v) ethanol.

The extract may refer to a product obtained by, regardless of an extraction method, an extraction solvent, an extracted component, or an extract type, and is also a broad concept that includes all materials that can be obtained by processing or treating the extract by using different methods such as fractionation, concentration, and the like after extraction.

In the composition including the Leonurus japonicus extract as the active ingredient, the extract may be included in an amount in a range of about 0.1 wt % to about 50 wt % based on the total weight of the composition.

The active ingredients included in the composition may be prepared as follows.

Although not limited thereto, the active ingredients may be prepared by the following three processes:

### First Process:

Step 1: After cleaning and shredding a dried Leonurus japonicus material, a solvent selected from water including purified water, C1-C4 lower alcohol including methanol, ethanol, or butanol, liquor, and a mixed solvent thereof at a volume about 1 time to about 20 times, preferably, about 4 times to about 8 times, the weight of the dried material, specifically, a mixed solvent of water and 20% to 40%(v/v) ethanol is mixed several times, and then, for example, at a temperature in a range of about 30° C. to about 150° C. or about 80° C. to about 120° C., for about, for example, 1 hour to about 48 hours or for about 8 hours to about 14 hours, an extraction method, such as ultrasonic extraction, hot-water extraction, room temperature-extraction, or reflux extraction, is utilized once to about 20 times, preferably, twice to 10 times repeatedly;

Step 2: The extract obtained from the step 1 is filtered, concentrated under reduced pressure, and dried to obtain a Leonurus japonicus extract in a dry state; and

Step 3: powder of the dried Leonurus japonicus extract obtained from the step 2 is mixed with one or more selected form the group consisting of the Lactobacillus plantarum KC3 strain (Accession No: KCTC13375BP) prepared according to a preparation method disclosed herein, the culture thereof, the fermentation solution thereof, the lysis solution thereof, the extract thereof, and the concentrate of the culture to form a mixture.

The term “respiratory disease” as defined herein refers to an inflammation disease of respiratory organs such as external nasal, nasal cavity, pharynx, trachea, bronchial tube, lungs, and the like. In detail, for example, the respiratory disease may include any one of respiratory inflammation diseases such as bronchitis, tuberculosis, chronic pulmonary disease, rhinitis, otitis media, viral respiratory disease, sore throat, tonsilitis, pneumonia, asthma, and chronic obstructive pulmonary disease (COPD). In more detail, the respiratory disease may include any one of respiratory inflammation diseases selected from the group consisting of bronchitis caused by air pollutants or fine dust, tuberculosis, chronic pulmonary disease, rhinitis, otitis media, viral respiratory disease, sore throat, tonsilitis, pneumonia, asthma, and COPD.

The term “prevention” as used herein refers to all actions that can inhibit or delay a respiratory disease by administering the composition including the extract above.

Also, the term “treatment” as used herein refers to all actions that can ameliorate or beneficially change symptoms of a respiratory disease by administering the composition.

The composition includes, as the active ingredients, the one or more selected from the group consisting of the Lactobacillus plantarum KC3 strain (Accession No: KCTC13375BP), the culture thereof, the fermentation solution thereof, the lysis solution thereof, the extract thereof, and the concentrate of the culture, and Leonurus japonicus extract, thereby reducing neutrophils that are inflammatory immune cells, inhibiting expression of one or more selected from the group consisting of bronchial inflammation factor cytokines IL-17A, TNF-α, and CXCL-1, and also reducing an amount of symmetricdimethylarginine (SDMA) in the blood of a patient having COPD.

The pharmaceutical composition including the active ingredients according to an aspect may be formulated in oral dosage form, such as a powder, granules, tablets, capsules, suspensions, emulsions, syrup, aerosol, and the like, in a suppository form, and in a sterilized injection solution form, each according to the methods in the art. Carriers, excipients, and diluents that may be included in the composition including the extract may include, for example, lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, and mineral oil. In the case of formulation, a commonly used diluent or excipient, such as a filler, a weighting agent, a binder, a wetting agent, a disintegrant, a surfactant, and the like may be used for preparation. Examples of solid formulations for oral administration are a tablet, a pill, a powder, a granule, a capsule, and the like. Such a solid formulation may be prepared by mixing the extract and fraction with at least one excipient, such as starch, calcium carbonate, sucrose or lactose, gelatin, and the like. Also, in addition to a simple excipient, lubricants, such as magnesium stearate and talc, may be used. Liquid preparation for oral administration may include suspension, emulsion, syrup, and the like, and in addition to commonly-used diluents such as water and liquid paraffin, various excipients such as wetting agents, flavorings, odorants, and preservatives may be included Formulations for parenteral administration may include a sterile solution, a non-aqueous solvent, a suspension, an emulsion, a freeze-dried agent, a suppository, and the like.

For the formulations above, a non-aqueous solvent and a suspension, such as propylene glycol, polyethylene glycol, vegetable oil including olive oil, injectable ester including ethyl oleate, and the like may be used. As a base agent for the suppository, witepsol, macrogol, tween 61, cacao butter, laurin butter, glycerol, gelatin, or the like may be used.

A dosage of the pharmaceutical composition according to an aspect may vary according to the condition and weight of a patient, the severity of disease, the drug form, and the route and duration of administration, but an appropriate effective amount may be selected by those skilled in the art. For the effects, the pharmaceutical composition including the active ingredients according to an aspect may be administered at an amount, for example, in a range of about 0.0001 mg/kg to about 100 mg/kg per day, and for example, about 0.001 mg/kg to about 100 mg/kg per day.

The pharmaceutical composition of the present disclosure may include the active ingredients at an amount, for example, in a range of about 0.1 wt % to about 50 wt % based on the total weight of the composition.

An aspect provides health functional food, a food composition, and a quasi-drug composition for preventing or ameliorating a respiratory disease, each including, as active ingredients, one or more selected from the group consisting of a Lactobacillus plantarum KC3 strain (Accession No: KCTC13375BP), a culture thereof, a fermentation solution thereof, a lysis solution thereof, an extract thereof, and a concentrate of the culture, and a Leonurus japonicus extract.

The term “health functional food” as defined herein indicates food manufactured and processed by using base materials or ingredients having functionality useful to humans, according to the Law for Health Functional Foods 6727 in South Korea. The term “functionality” as used herein indicates ingestion to adjust nutrients with regard to a structure and functions of a human body or obtain effects advantageous to health care such as physiological effects. Therefore, the health functional food of the present disclosure refers to a food group that gives added values to act and express functions of food for a specific purpose by using physical, biochemical, and bioengineering methods on the food, or food designed and processed to sufficiently express functions of the food composition, such as regulating the biological defense rhythm and body control functions associated with disease prevention and recovery, to the living body. In this regard, the health functional food of the present disclosure refers to food that can sufficiently express the bioregulatory functions of preventing or ameliorating a respiratory disease. The health functional food may include a food supplementary additive that is pharmaceutically acceptable, and may further include a carrier, an excipient, and a diluent that are commonly used in the preparation of health functional food.

As food to which the composition of the present disclosure can be added may include, for example, various types of food, beverage, gum, candy, tea, vitamin complex, functional food, and the like. In addition, in the present disclosure, food may include special nutritious food (e.g., infant formula milk, infant/baby food, etc.), processed meat products, fish meat products, tofu, jellied food, noodles (e.g., ramen, noodles, etc.), health supplement food, seasoned food (e.g., soy sauce, soybean paste, red pepper paste, mixed soy paste, etc.), sauces, confectionaries (e.g., snacks), dairy products (e.g., fermented milk, cheese, etc.), other processed foods, kimchi, pickled food (e.g., various types of kimchi, pickles, etc.), beverages (e.g., fruit juice, vegetable juice, soy milk, fermented beverages, ice cream, etc.), natural seasonings (e.g., ramen powder, etc.), vitamin complexes, alcoholic beverages, alcoholic liquors, and other health supplement food, but is not limited thereto. The food, beverages, or food additives may be prepared according to the preparation methods in the art.

In addition to those described above, the food composition according to an aspect may include various types of nutrients, vitamins, minerals (e.g., an electrolyte), flavorings, such as synthetic and natural flavorings, coloring agents, improving agent (e.g., cheese, chocolate, etc.), pectic acid and a salt thereof, alginic acid and a salt thereof, organic acid, protective colloidal adhesive agents, pH regulators, stabilizers, preservatives, glycerin, alcohol, carbonizing agents used in carbonate beverages, and the like. These components may be used independently or in combination, and the proportion of these additives may also be appropriately selected by those skilled in the art.

In the health functional food for preventing or ameliorating a respiratory disease, the extract may be included at an amount in a range of about 0.01% to about 95%, preferably, about 1% to about 80%, based on the total weight of the composition.

In addition, the composition including, as the active ingredients, one or more selected from the group consisting of the Lactobacillus plantarum KC3 strain (Accession No: KCTC13375BP), the culture thereof, the fermentation solution thereof, the lysis solution thereof , the extract thereof, and the concentrate of the culture, and the Leonurus japonicus extract, for preventing or ameliorating a respiratory disease may be prepared and processed for use as health function food in the pharmaceutical dosage forms, such as powder, granules, tablets, capsules, pills, suspensions, emulsion, syrup, and the like, or as health functional food in the form of tea bags, leached teas, and health drinks.

In addition, the health functional food may additionally include a food additive, and the suitability as “food additives” is determined according to standards and criteria of items in accordance with the general provisions and general analytical method of the Korean Food Additive Code approved by the Ministry of Food and Drug Safety, unless otherwise specified.

Examples of products listed in the “Korean Food Additive Code” may include ketones, chemical products such as glycine, potassium citrate, nicotinic acid and cinnamic acid, natural additives such as a persimmon color, licorice extract, crystalline cellulose and guar gum, and mixed formulations such as monosodium L-glutamate, alkali agents for noodles, preservative formulation and tar color formulation.

Examples of the functional food according to an aspect, including, as the active ingredients, one or more selected from the group consisting of the Lactobacillus plantarum KC3 strain (Accession No: KCTC13375BP), the culture thereof, the fermentation solution thereof, the lysis solution thereof, the extract thereof, and the concentrate of the culture, and the Leonurus japonicus extrac, may include confectionary such as bread, rice cake, dried fruit, candy, chocolate, chewing gum, and jam, ice cream products such as ice cream, frozen dessert, and ice cream powder, dairy products such as milk, low-fat milk, lactose-free milk, processed milk, goad milk, fermented milk, buttermilk, condensed milk, milk cream, butter oil, butter oil, natural cheese, processed cheese, milk powder, and whey, meat products such as processed meat products, egg products, and hamburger, fish and meat products including processed fish and meat products such as fish cake, ham, sausage, and bacon, noodles such as instant noodle, dried noodle, raw noodle, instant fried noodle, instant non-fried noodle, processed noodle, frozen noodle, pasta, fruit drink, vegetable drink, carbonated drink, soy milk, lactic acid beverage such as yogurt, beverage such as mixed drink, seasonings such as soy sauce, soybean paste, red pepper paste, black soybean paste, fermented soybean paste, mixed soy paste, vinegar, sauces, tomato ketchup, curry, and dressing, margarine, shortening, and pizza. However, embodiments of the present disclosure are not limited thereto.

The health functional beverage composition according to an aspect has no particular limitation on components other than the inclusion of the strain above as an essential ingredient at an indicated ratio, and may additionally include various flavorings or natural carbohydrates as in the existing beverages. Examples of the above natural carbohydrates include general sugar such as monosaccharides (e.g., glucose, fructose, etc.), disaccharides (e.g., maltose, sucrose, etc.), and polysaccharides (e.g., dextrin, cyclodextrin, etc.), and sugar alcohols such as xylitol, sorbitol, and erythritol. In addition to those described above, natural flavorings (thaumatin, stevia extracts (e.g., rebaudioside A, glycyrrhizin, etc.)), and synthetic flavoring agents (e.g., saccharin, aspartame, etc.) may be advantageously used as flavorings. A ratio of the natural carbohydrates may be typically in a range of about 1 g to about 20 g, preferably, about 5 g to about 12 g, per 100 ml of the composition of the present disclosure.

In addition to those described above, the food composition according to an aspect may include various types of nutrients, vitamins, minerals (e.g., an electrolyte), flavorings, such as synthetic and natural flavorings, coloring agents, improving agent (e.g., cheese, chocolate, etc.), pectic acid and a salt thereof, alginic acid and a salt thereof, organic acid, protective colloidal adhesive agents, pH regulators, stabilizers, preservatives, glycerin, alcohol, carbonizing agents used in carbonate beverages, and the like. The other components than the aforementioned components may be fruit pulp for preparing natural fruit juice, a fruit juice beverage, and vegetable juice. Such components may be used independently or in combination. A ratio of the additives is not so important, but is generally selected in a range of about 0.1 part by weight to about 20 parts by weight based on 100 parts by weight of the composition of the present disclosure.

In addition, the active ingredients according to an aspect may be added to food or beverages for the prevention of purposed diseases. In this case, the amount of the mixture of the strain and the extract in food or beverages may be in a range of about 0.01 wt % to about 15 wt % based on the total weight of the food, and the health beverage composition may be added at a ratio in a range of about 0.02 g to about 5 g, preferably about 0.3 g and to about 1g, per 100 ml

During a process of preparing the health functional food, the amount of the mixture according to an aspect that is added to food including beverages may be appropriately adjusted according to necessity.

The term “quasi-drug” as used herein refers to one of: textiles, rubber products, or the like used for the purpose of treating, alleviating, treating, or preventing diseases of humans or animals; materials that have weak action on the human body or do not act directly on the human body, and that are anything other than appliances or devices; and preparations that are used for sterilization, insecticide, and similar purposes to prevent infection. The quasi-drug may also refer to: items excluding those other than an instrument, a machine, or an apparatus used for the purpose of diagnosing, treating, alleviating, handling, or preventing conditions or diseases in humans or animals; and items excluding those other than an instrument, a machine, or an apparatus used for the purpose of pharmacologically affecting the structure and function of humans or animals. The quasi-drug may also include skin external preparations and personal care products.

Another aspect provides a method of preventing or treating a respiratory disease, the method including administering a composition to a subject in need thereof, the composition including, as active ingredients, one or more selected from the group consisting of a Lactobacillus plantarum KC3 strain (Accession No: KCTC13375BP), a culture thereof, a fermentation solution thereof, a lysis solution thereof, an extract thereof, and a concentrate of the culture, and a Leonurus japonicus extract.

As another aspect, the present disclosure provides use of a composition for the treatment of a respiratory disease, the composition including, as active ingredients, one or more selected from the group consisting of a Lactobacillus plantarum KC3 strain (Accession No: KCTC13375BP), a culture thereof, a fermentation solution thereof, a lysis solution thereof, an extract thereof, and a concentrate of the culture and a Leonurus japonicus extract.

The composition according to an aspect including, as the active ingredients, the Leonurus japonicus extract and the one or more selected from the group consisting of the Lactobacillus plantarum KC3 strain (Accession number: KCTC13375BP), the culture thereof, the fermentation solution thereof, the lysis solution thereof, the extract thereof, and the concentrate of the culture may be administered to a subject in need thereof. The term “subject” as used herein refers to a target in need of treatment for a disease, and more particularly, to mammals including humans or non-humans, such as primates, rodents (e.g., rat, mice, guinea pigs, etc.), mice, dogs, cats, horses, cattle, sheep, pigs, goats, camels, and antelopes, each having a respiratory disease.

The pharmaceutical composition may be administered to a subject by various methods known in the art, such as intraperitoneal administration, intravenous administration, intramuscular administration, subcutaneous administration, intradermal administration, oral administration, topical administration, intranasal administration, intrapulmonary administration, rectal administration, and the like, but embodiments of the present disclosure are not limited thereto.

Also, regarding the method, the effective amount (or dosage) of the composition of an aspect may be, for example, in a range of about 0.0001 mg to about 10,000 mg, about 0.001 mg to about 1,000 mg, about 1.0 mg to about 100 mg, about 0.01 mg to about 1,000 mg, about 0.01 mg to about 100 mg, about 0.01 mg to about 10 mg , or about 0.01 mg to about 1 mg. The administration may be performed once a day or several times a day. The administration dosage does not limit the scope of the present disclosure in any aspect.

### Advantageous Effects of Disclosure

The composition according to an aspect, including, as the active ingredients, the Lactobacillus plantarum KC3 strain (Accession No: KCTC13375BP) and the Leonurus japonicus extract may have a defense effect against respiratory damage caused by air pollutants such as fine dust and can inhibit expression of IL-17A, TNF-α, and CXCL-1, thereby being able to effectively treat or prevent a respiratory disease including chronic obstructive pulmonary disease (COPD). In addition, the effect of the active ingredient combination above results in a synergistic inhibition or treatment effect on bronchial inflammation by the administration compared to the existing therapeutic effect of a respiratory inflammatory disease of each of the Leonurus japonicus extract and the lactic acid bacteria KC3. Thus, the present disclosure can be usefully utilized for the prevention or treatment of a respiratory disease.

### BRIEF DESCRIPTION OF DRAWINGS

FIG. 1 shows a deposit certificate of Lactobacillus plantarum KC3;

FIG. 2 shows an experiment result on bile tolerance against a Lactobacillus plantarum KC3 strain (wherein all numerical values (or data) are the mean±standard deviation of 3 repetitions, and * represents a case of p<0.05 between a group with oxgall and a group without oxgall);

FIG. 3 shows an experiment result on pH resistance against the Lactobacillus plantarum KC3 strain;

FIG. 4 is a diagram confirming the total number of cells included in bronchoalveolar lavage (BAL) fluid by performing BAL in which each sample (the KC3 strain, the Leonurus japonicus extract, or a mixture thereof, and dexamethasone for treating a positive control group) is treated or not treated on a mouse model having respiratory damage (Normal: normal control group, control: sample untreated group, PC: positive control group, KC3: KC3 strain alone, Leo: Leonurus japonicus extract alone, KC3+Leo: mixed administration group of lactic acid bacteria and Leonurus japonicus extract); and

FIG. 5 is a diagram confirming inhibition rates of increase in the cell number in BAL fluid in a group in which each sample (the KC3 strain, the Leonurus japonicus extract, or the mixture thereof, and positive control group treated with dexamethasone) for a mouse model having respiratory damage, compared to the respiratory injury-induced group (PC: positive control group, KC3: KC3 lactic acid bacteria alone, Leo: Leonurus japonicus alone, KC3+Leo: Mixed administration of lactic acid bacteria and Leonurus japonicus extract).

### MODE OF DISCLOSURE

Hereinafter, preferable Examples and Experimental Examples are provided to help understanding of the present disclosure. However, Examples below are only provided for easier understanding of the present disclosure, and the content of the present disclosure is not limited by Examples and Experimental Examples below.

### EXAMPLE 1

### Isolation of New Lactic Acid Bacteria from Lactobacillus plantarum KC3

### EXAMPLE 1-1

### Preparation of Raw Materials of Kimchi

For use as cabbage kimchi which is a raw material of the present disclosure, kimchi for family use in North Jeolla Province was prepared according to the following process using materials all purchased from a local mart (Hanaro Mart, Wansan-gu, Jeonju-si).

(1) Step 1: Five Korean cabbages (about 1 kg each) were prepared and cut into two pieces after getting rid of inedible portions. 500 g of salt was dissolved in water in a container for salting. The divided Korean cabbage pieces were soaked in salted water, and after being taken out of the salted water, salt was sprinkled in layers between cabbage leaves. The Korean cabbages were then salted for 5 to 6 hours, washed with clean water 3 to 4 times, and placed on a large colander for dehydration.

(2) Step 2: Two Korean radishes (about 1.2 kg each) were prepared, trimmed and washed after getting rid of radish leaves, and then cut into thin strips in 4-5 cm long. Half bundle of great green onions (about 0.5 kg), half bundle of chives (about 0.5 kg), and half bundle of mustard leaves (about 0.5 kg) were also trimmed and washed, and cut into the same length as the radish strips.

(3) Step 3: 50 g of garlic, 10 g of ginger, and 200 g of salted shrimp were finely minced, and 300 ml (about 200 g) of anchovy sauce was prepared. Rice porridge was cooked with 150 g of glutinous rice soaked in water. After cooling the rice porridge, the prepared anchovy sauce and the minced salted shrimp, garlic, and ginger were added thereto with 500 g of red pepper powder, and all the seasonings were mixed evenly.

(4) Step 4: After putting and mixing all the radish strips, great green onion, chives, and mustard leaves that were all cut to the similar length as in Step 2, kimchi seasoning was made by seasoning with salt (about 0.5 kg) and sugar (about 0.5 kg).

(5) Step 5: After spreading the kimchi seasoning evenly between the Korean cabbage leaves, the Korean cabbage was rolled by the outermost leaf. Then, the Korean cabbage was placed one by one in a container in a way that the cross section of the Korean cabbage faced up, and the container was stored in a low-temperature storage (0° C. to −2° C.) so as to ripen the kimchi for 1 year, thereby producing raw materials for the Korean cabbage kimchi.

### EXAMPLE 1-2

### Isolation and Identification of New Lactobacillus plantarum KC3 Strain

Regarding isolation and identification processes for the Lactobacillus plantarum KC3 strain of the present disclosure, the raw materials for the Korean cabbage kimchi of Example 1-1 were inoculated by 0.1 ml each onto an MRS sodium medium (supplemented with MRS medium (DF0881-17-5, Difco) and 1.5% agar (214010, Difco)) to which bromcresol purple (114375, Sigma) and sodium azide (S2002, sigma) were diluted with a peptone diluent (MB-B2220, MB cell) and added, by a streak-plate method. After culturing in an anaerobic condition at 37° C. for 48 hours, colonies that turned yellow in the medium were selected as tentative lactic acid bacteria.

As a result of identifying the isolated strain, it was confirmed that the strain was a gram-positive facultative anaerobic bacillus was negative for the catalase and motility.

It was also confirmed that the strain did not grow at 15° C. and 45° C., and based on that no gas from glucose and no ammonia from alginine were produced, the strain was confirmed to belong to the genus Lactobacillus.

### EXAMPLE 1-3

### Identification of Microorganism (Based on Analysis of Glucose Utilization and 16s rRNA Identification)

1-3-1. Analysis of Glucose Utilization

The glucose utilization of the selected lactic acid bacteria was analyzed using an API CHL50 kit (50300, bioMerieux). As a result of the analysis, it was confirmed that, as shown in Table 1 below, glucose from D-ribose, D-galactose, D-glucose, D-fructose, D-mannose, D-mannitol, D-sorbitol, methyl-αD-mannopyranoside, amyglandine albutine, esculin ferric citrate, D-cellobiose, D-maltose, D-lactose, D-melibiose, D-saccharose, D-trehalose, D-melezitose, and D-raffinose was utilized.

TABLE 1
Glucose utilization of Lactobacillus plantarum KC3

| | uti- | | uti- |
|---|---|---|---|
| Glucose | lization | Glucose | lization |
| Control | − | Esculin ferric citrate | + |
| Glycerol | − | Salicin | ± |
| Erythritol | − | D-Celiobiose | + |
| D-Arabinose | − | D-Maltose | + |
| L-Arabinose | − | D-Lactose | + |
| D-Ribose | + | D-Melibiose | + |
| D-Xylose | − | D-Saccharose | + |
| L-Xylose | − | D-Trehalose | + |
| D-Adonitol | − | Inulin | − |
| Methyl-βD-Xylopyran- | − | D-Melezitose | + |
| oside | | | |
| D-Galactose | + | D-Raffinose | + |
| D-Glucose | + | Amidon | − |
| D-Fructose | + | Glycogen | − |
| D-Mannose | + | Xylitol | − |
| L-Sorbose | − | Gentiobiose | ± |
| L-Rhamnose | − | D-Turanose | − |
| Dulcitol | − | D-Lyxose | − |
| Inositol | − | D-Tagatose | − |
| D-Mannitol | + | D-Fucose | − |
| D-Sorbitol | + | L-Fucose | − |
| Methyl-αD-Mannopyran- | + | D-Arabitol | − |
| oside | | | |
| Methyl-αD-Glucopyran- | − | L-Arabitol | − |
| oside | | | |
| N-AcetylGlucosamine | ± | potassium Gluconate | − |
| Amygdalin | + | potassium 2-KetoGluconate | − |
| Arbutin | + | potassium 5-KetoGluconate | − |

1-3-2. 16s rRNA Identification

The colonies grown on the MRS solid medium (supplemented with MRS medium (DF0881-17-5, Difco) and 1.5% agar (214010, Difco)) were collected and subjected to double-stranded DNA sequencing (Solgent, Korea). As a result of identifying the strain by BLAST, the obtained nucleic sequence (SEQ ID NO: 1 in Table 2) showed a homology of 99% to the Lactobacillus plantarum, confirming that the new microorganism of the present disclosure was the strain (hereinafter also referred to as “new lactic acid bacteria from KC3” or “CKDB-KC3”).

TABLE 2
16s rRNA nucleic sequence of
Lactobacillus plantarum KC3

| 16s rRNA nucleic sequence of | SEQ |
|---|---|
| Lactobacillus plantarum KC3 | ID NO. |
| AGATTAGACGTTCCCTTCGGGGACATGGATACAGGTGGTGC | 1 |
| ATGGTTGTCGTCAGCTCGTGTCGTGAGATGTTGGGTTAAGT | |
| CCCGCAACGAGCGCAACCCTTATTATCAGTTGCCAGCATTA | |
| AGTTGGGCACTCTGGTGAGACTGCCGGTGACAAACCGGAGG | |
| AAGGTGGGGATGACGTCAAATCATCATGCCCCTTATGACCT | |
| GGGCTACACACGTGCTACAATGGATGGTACAACGAGTTGCG | |
| AACTCGCGAGAGTAAGCTAATCTCTTAAAGCCATTCTCAGT | |
| TCGGATTGTAGGCTGCAACTCGCCTACATGAAGTCGGAATC | |
| GCTAGTAATCGCGGATCAGCATGCCGCGGTGAATACGTTCC | |
| CGGGCCTTGTACACACCGCCCGTCACACCATGAGAGTTTGT | |
| AACACCCAAAGTCGGTGGGGTAACCTTTTAGGAACCAGCCG | |
| CCTAAGGTGGGACAGATGATTAGGGTGAAGTCGTACA | |

1-3-3. Characteristics of Microorganism

The characteristics of the new Lactobacillus plantarum KC3 according to the present disclosure are as follows:

(1) Form of Bacteria

Form of bacteria when cultured in MRS agar plate medium at 37° C. for 48 hours

\textcircled{1} Cell type: Bacillus

\textcircled{2} Mobility: None

\textcircled{3} Spore-forming ability: None

\textcircled{4} Gram staining: Positive

(2) Shape of Colony

Form of colonies when cultured in MRS agar plate medium at 37° C. for 48 hours

\textcircled{1} Shape: Round

\textcircled{2} Bulge: Convex

\textcircled{3} Surface: Smooth

\textcircled{4} Color: Milky-white

(3) Physiological Properties

\textcircled{1} Temperature for growth and development

Temperature enable growth and development: 15° C. to 40° C.

Optimal temperature for growth and development: 36° C. to 38° C.

\textcircled{2} pH for growth and development

pH enable growth and evelopment: 4.6 to 7.5

Optimal pH: 6.0 to 7.0

\textcircled{1} Effect on oxygen: Facultative anaerobic

(4) Catalase: Negative

(5) Gas generation: Negative

(6) Indole production: Negative

(7) Lactic acid production: Positive

(8) Biogenic amine production: Negative

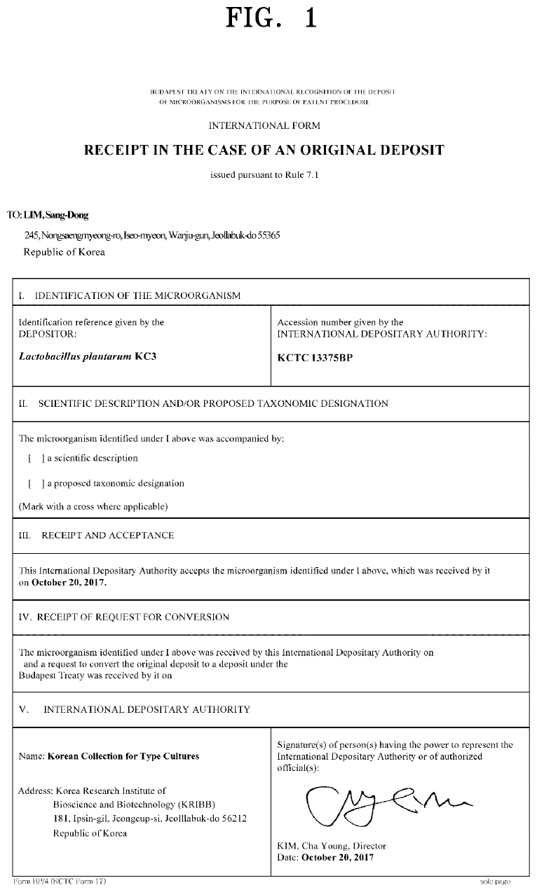

Based on the results of the microorganism identification and the bacteria characteristics above, a new strain isolated from kimchi was named Lactobacillus plantarum KC3 and deposited at the Korea Research Institute of Bioscience and Biotechnology (Accession No: KCTC13375BP) on Oct. 20, 2017 (FIG. 1).

### EXAMPLE 2

### Confirmation of Characteristics of New Lactic Acid Bacteria from Lactobacillus plantarum KC3

### EXAMPLE 2-1

### Tolerance Experiment of Gastric Acid and Bile Acid

Gastric acid secretion in gastric fluids and bile acid secreted from the pancrease are significantly important factors affecting the survival of microorganisms. Thus, in order to confirm gastric acid-resistance and bile acid-resistance of the new lactic acid bacteria from the Lactobacillus plantarum KC3 of the present disclosure, an experiment was performed as follows.

It was a process of examining the resistance to artificial gastric fluids and bile to explore the possibility for use as probiotics, and then selecting and identifying strains having excellent activity and strong resistance.

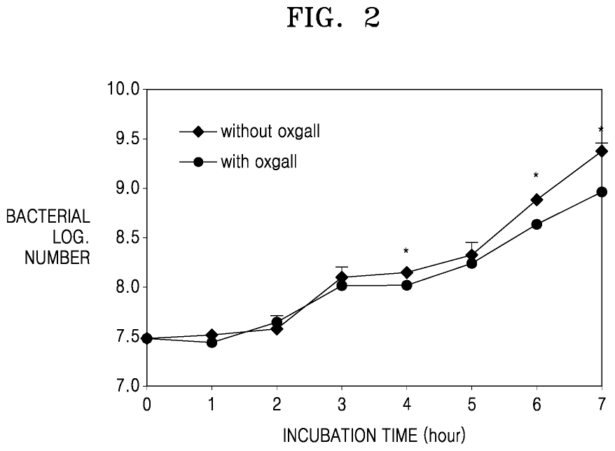

FIG. 2 is a diagram showing the results of the bile-resistance test against the Lactobacillus plantarum KC3 strain of the present disclosure.

More specifically, the Lactobacillus plantarum KC3 strain was grown in an MRS medium (with oxgall) containing 0.03% of bile (oxgall) and 0.05% of L-cysteine and an MRS medium (without oxgall) containing 0.05% of L-cysteine. All values (or data) are the mean±standard deviation for triplicate experiments, and * indicates a case where p<0.05 between a group including oxgall and a group not including oxgall.

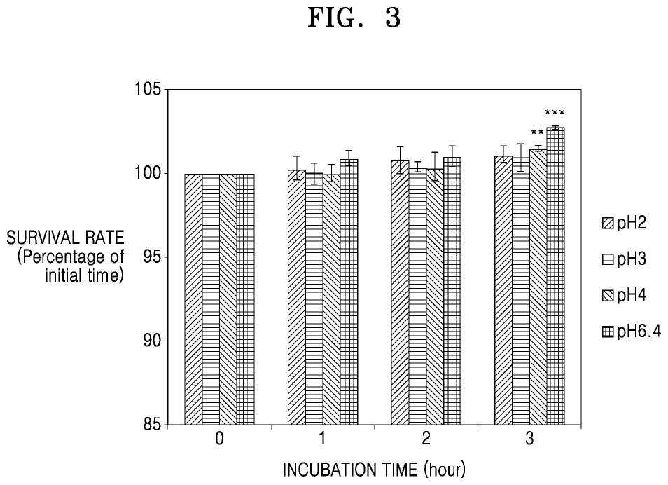

FIG. 3 is a diagram showing the results of pH resistance test against the Lactobacillus plantarum KC3 strain of the present disclosure.

More specifically, the figure shows the survival rate of the Lactobacillus plantarum KC3 strain after 3 hours in hydrochloric acid solution having a pH of 2.0, 3.0, 4.0 and 6.4, and as compared with the start point (or start time), * indicates a case of p<0.05, ** indicates a case of p<0.01, and *** indicates a case of p<0.001.

### EXAMPLE 2-2

### Confirmation of Antibacterial Activity of Strain

In order to confirm antibacterial activity of the new lactic acid bacteria from the Lactobacillus plantarum KC3, an antibacterial activity experiment was performed.

The antibacterial activity experiment was to confirm the inhibitory activity against Escherichia coli, Salmonella typhimurium, Staphylococcus aureus, and Listeria monocytogenes. The stronger the inhibitory activity against harmful bacterial is, the better the antibacterial activity is.

Table 3 shows the experimental results about the antibacterial activity of the Lactobacillus plantarum KC3 strain, wherein the initial number of bacteria of the Lactobacillus plantarum KC3 strain was about 2.10±0.17×10⁶ CFU/mL, and the results were obtained after 6 hours of the experiments at 37° C. Here, all values (or data) are the mean±standard deviation for triplicate experiments.

TABLE 3
Antibacterial activity of Lactobacillus plantarum KC3

| | Growth | | | | |
|---|---|---|---|---|---|
| | pathogens^{a} | | KC3 + pathogens^{a} | | Inhibition |
| Pathogens | CFU/mL | pH | CFU/mL | pH | (%) |
| Escherichia coli | 3.23 ± 0.25 × 10⁶ | 5.98 | 8.50 ± 0.05 × 10⁵ | 4.84 | 73.98% |
| Salmonella | 6.46 ± 0.35 × 10⁶ | 6.10 | 4.00 ± 0.26 × 10⁶ | 5.25 | 38.14% |
| Typhimurium | | | | | |
| Listeria | 1.57 ± 0.20 × 10⁵ | 6.06 | 1.13 ± 0.06 × 10⁵ | 4.94 | 27.97% |
| monocytogenes | | | | | |
| Staphyloccous aureus | 3.46 ± 0.87 × 10⁶ | 6.08 | 2.83 ± 0.61 × 10⁶ | 4.9 | 18.27% |

### EXAMPLE 2-3

### Experiment on Antibiotic Susceptibility of Strain

In order to confirm antibiotics resistance level of the new lactic acid bacteria from the of the Lactobacillus plantarum KC3 the present disclosure, an experiment was performed as follows by applying the method described in the document

To measure the antibiotics resistance level of the strain, an MIC test was carried out. Lactic acid bacteria that were inoculated into an MRS medium (DF0881-17-5, Difco) and cultured at 37° C. for 18 hours were spread on an LSM solid medium (90% iso-sensitest broth (CM0473, Oxoid), 10% MRS medium (DF0881-17-5, Difco), and 1.5% agar (214010, Difco)). Strips for each type of antibiotics, such as Amikacin (92018, Liofilchem srl), Gentamycin (92009, Liofilchem srl), Kanamycin (92034, Liofilchem srl), Streptomycin (92112, Liofilchem srl), Penicillin-G (92102, Liofilchem srl), Oxacillin (92015, Liofilchem srl), Ampicillin (920030, Liofilchem srl), Bacitracin (92019, Liofilchem srl), Rifampicin (92001, Liofilchem srl), Polymyxin B (92004, Liofilchem srl), Chloramphenico 1(92075, Liofilchem srl), Vancomycin (92057, Liofilchem srl), and the like, were put on the medium, and the bacteria were grown at 37° C. for 24 hours. Then, a section where a clear zone disappeared was observed with the naked eyes, and an MIC was measured therefrom.

Table 4 shows the results of the antibiotics resistance against the Lactobacillus plantarum KC3 strain. In Table 4, R indicates resistance and represents that the size of an inhibition zone is about 0 mm; IS indicates mediate resistance and represents that the size of an inhibition zone is in a range of about 1 mm to about 5 mm; and S indicates susceptibility and represents that the size of an inhibition zone is greater than about 5 mm.

TABLE 4
Antibiotics susceptibility of Lactobacillus plantarum KC3

| Anti-microbial | Antibiotic | Anti-microbial | Antibiotic |
|---|---|---|---|
| agents | resistance | agents | resistance |
| Aminoglycosides | | Gram-positive-spectrum | IS (4 mm) |
| Amikacin | IS (1 mm) | Bacitracin | S (7 mm) |
| Gentamycin | IS (3 mm) | Rifampicin | S (7 mm) |
| Kanamycin | R (0 mm) | Novabiocin | S (7 mm) |
| Neomycin | IS (3 mm) | Lincomycin | S (10 mm) |
| Streptomycin | R (0 mm) | Gram-negative spectrum | |
| β-lactams | | Polymyxin B | R (0 mm) |
| Penicillin-G | IS (5 mm) | Broad spectrum | |
| Oxacillin | IS (2 mm) | Chloramphenicol | S (10 mm) |
| Ampicillin | S (14 mm) | Vancomycin | R (0 mm) |

### EXAMPLE 2-4

### Confirmation of Biogenic Amine Producibility of Strain

To confirm biogenic amine producibility of the new lactic acid bacteria from the Lactobacillus plantarum KC3 of the present disclosure, an experiment was performed as follows by applying the method described in the document

Biogenic amines are produced by fermentation of food and may vary depending on a type of microorganisms or chemical and physical conditions. Since biogenic amines produced in fermented food can cause food poisoning or allergic reactions, the biogenic amines are considered as important criteria for selecting a safe strain for food engineering

Accordingly, to confirm whether the strain of the present disclosure formed biogenic amines, the strain grown in an MRS liquid medium (DF0881-17-5, Difco) for 16 hours at 37° C. was transferred to a special medium and cultured at 37° C. for 48 hours.

An MRS liquid medium (DF0881-17-5, Difco) to which an amino acid precursor for each of tyrosine (SIGMA, T1145), histidine (SIGMA, H5659), ornithine (SIGMA, 02375), and lysine (DAEJUNG, 5093-4105) was added was prepared. In each medium, it was confirmed whether the biogenic amines, i.e., tyramine, histamine, putrescine, and cadaverine, were produced by the strain. In detail, onto an MRS liquid medium (DF0881-17-5, Difco) to which 0.1% of the amino acid precursor was added, 1% of the isolated Lactobacillus plantarum strain was inoculated, and then subcultured 5 times to 10 times. The resulting strain was then spread on a biogenic amine identification medium [prepared by mixing 0.5% of trypton, 0.5% of yeast extract, 0.5% of cocoon extract, 0.5% of sodium chloride, 0.25% of glucose, 0.05% of Tween-80, 0.02% of magnesium sulfate, 0.005% of manganese sulfate, 0.004% of iron sulfate, 0.2% of citric acid salt, 0.001% of thiamine, 0.2% of K2PO4, 0.01% of calcium carbonate, 0.005% of pyridoxal-5-phosphate, 1% of amino acid, 0.006% of bromocresol purple, and 2% of agar with distilled water and adjusting the pH to 5.3 for use], and cultured at 37° C. for 24 hours to 48 hours. Then, by checking whether the color changes to purple, the biogenic amine producibility of the strain was determined.

Bromocresol purple contained in a decarboxylase medium is yellow at pH 5.2, but turns purple as the pH increases to 6.8. Thus, based on the color that turns purple as the pH increases by the production of the biogenic amines, the production of the biogenic amines was confirmed.

Table 5 below shows the results of analyzing the biogenic amine producibility of the Lactobacillus plantarum KC3 strain. As shown in Table 4, it was confirmed that the strain was negative for all of putrescine, tyramine, histamine, and cadaverine. Accordingly, it was confirmed that the strain of the present invention had no ability to produce the biogenic amines that can induce hypersensitive immune responses.

TABLE 5
Biogenic amine producibility of Lactobacillus plantarum KC3

Biogenic amines

| Strain | Putrescine | Tyramine | Histamine | Cadaverine |
|---|---|---|---|---|
| KC3 | − | − | − | − |

### EXAMPLE 3

### Culture and Preparation of Lactic Acid Bacteria from Lactobacillus plantarum KC3

Culturing of the isolated and identified Lactobacillus plantarum KC3 was performed in a flask containing an MRS medium (supplemented with an MRS (DF0881-17-5, Difco)) associated with lactic acid bacteria seed at 37° C. for 24 hours.

Each culture was inoculated into an optimized medium (self-manufactured) in a fermenter (Bio Control & Science, MARADO-05D-PS).

For the fermentation, the pH was maintained constant between 5.5 and 6.0 by automatically adding NaOH solution (25% w/v) to the medium, and the fermentation was performed at 37° C. for 18 hours to 20 hours while stirring at 120 rpm.

Lyophilization of 40× concentrated cells was performed according to the manual (Cooling &amp; Heating System, Lab-Mast 10).

After lyophilization, colony-forming units (CFU) per 1 g of each probiotic powder were measured by serial dilution. The strain was suspended in 0.1 M PBS, and the density thereof was adjusted to 10⁹ CFU/mL before use.

### EXAMPLE 4

### Preparation of Leonurus japonicus Extract

500 g of dried whole herb of Leonurus japonicus (available by Human herb) was evenly mixed, and 30% alcoholic liquor mixed with distilled water was added thereto. First extraction process was performed thereon at 80±2° C. for 4 hours, and then, a filtration process was repeated twice with a filtering paper having a size of 1 μm. The extract thus obtained was then concentrated in a vacuum at 52.5±2.5° C. and about 650±30 mmHg. Following sterilization at 85.0±2.0° C. for 1 hour, the resultant product was cooled to 55° C., and a spray drier (KL-8,Seokang Engineering Inc., inlet temperature of 190±10° C., outlet temperature of 95±5° C.) was used to prepare a Leonurus japonicus extract(71 g, hereinafter referred to as “LS”).

### EXAMPLE 5

### Preparation of Cmposition Icluding, as Active Ingredients, Lactobacillus plantarum KC3 Strain and Leonurus japonicus Extract

6.7 mg/mL (=1×10⁹ CFU/cell) of the Lactobacillus plantarum KC3 strainof Example 1 and 8.3 mg/mL(=100 mg/kg BW) of the Leonurus japonicus extract solution of Example 4 were mixed ata ratio of 1:1 based on the dry weight, so as to prepare a mixture (KC3+Leo) of the Lactobacillus plantarum KC3 strain and the Leonurus japonicus extract.

Experimental Example 1

### Confirmation of Defense Effect of Mixture of Lactobacillus plantarum KC3 strain and Leonurus japonicus Extract Against Respiratory Damage Caused by Air Pollutants

1-1. Experiment on Mouse Model having Respiratory Damage

An experiment was carried out to confirm whether the composition prepared in Examples above exhibited a defense effect against respiratory damage caused by air pollutants. BALB/c male mice (7-week-old, 20 g to 25 g, Orient Bio) were divided into groups of 6 mice, and as assigned to 6 mice in each group, and Alum was diluted in components of air pollutants, i.e., 10 mg/ml of coal combustible materials, 10 mg/ml of fly ash, and 5 mg/ml of diesel exhaust particle (DEP) to have a final concentration of 1% so that a final concentration of each component was 1.5 mg/ml for coal combustible material/fly ash and 5 mg/ml for DEP in a mixture. Then, to all groups except for a normal group, the mixture was directly injected into the airway and nose of the experimental animals by 50 pl each on the 4^{th}, 7^{th} and 10^{th} says of the experiment.

For a positive control group treated with dexamethasone (Sigma D2915), administed at 3 mg/kg BW of the mouse model, the Lactobacillus plantarum KC3 strain (KC3) was diluted at a concentration of 1×10⁹CFU/cell (about 2 mg/kg BW), the Leonurus japonicus extract was diluted in distilled water at a dose of 100 mg/kg of BW, and then orally administered at a dose of 300 pl every day (for 11 days). For a mixed group including the KC3 strain and the Leonurus japonicus extract, the solution was mixed and diluted at a ratio of 1:1 by the dry weight as in Examples above, and then orally administered at a dose of 300 pl every day (for 11 days) (the cell amount in the KC3 included in the mixture was 0.5×10⁹ CFU/cell, and the dose of the Leonurus japonicus extract was set at 50 mg/kg BW). The oral administration was performed at a dose of 300 μl per mouse, and an autopsy was performed on the 12^{th} day after the start of the experiment to recover the BAL solution.

### Experimental Example 1-2

### Confirmation of Total Number of Cells in BAL Fluid

In the case of treatment with the Lactobacillus plantarum KC3 strain, the Leonurus japonicus extract, or the mixture thereof, BAL was performed in a disease mouse model by the following method to confirm how the total number of cells in BAL fluid changed (see Schins et al., Toxicol Appl Pharmacol. 195(1), 1-11 (2004) and Smith et al., Toxicol Sci, 93(2), 390-399 (2006)).

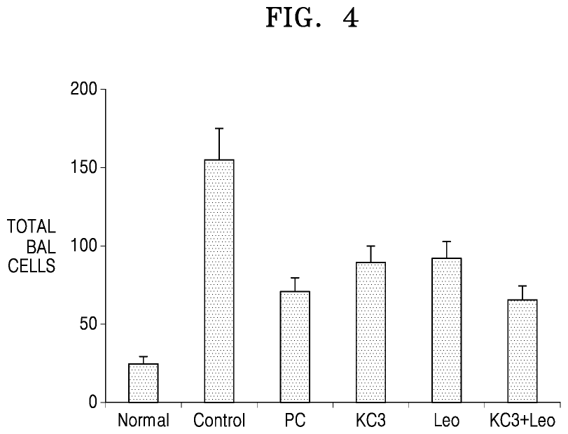

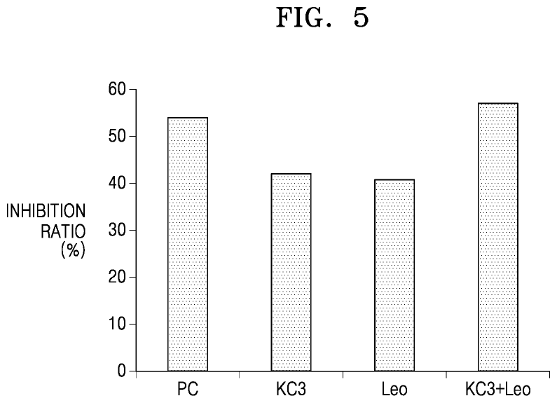

In the case of treatment with the Lactobacillus plantarum KC3 strain, the Leonurus japonicus extract, or the composition of Examples above, a BAL experiment was performed in a disease mouse model. Results obtained by confirming the total number of cells in the BAL fluid are shown in Table 6 and FIGS. 4 and 5.

TABLE 6
Changes in the total number of cells in BAL fluid

| | Total number of | Inhibition rate |
|---|---|---|
| | BAL cells (×10⁴ | (based on |
| Division | cells/ml) | induced group) |
| Normal control group | 25.5 ± 4.1 | |
| Induced group | 155.3 ± 19.4 | |
| Positive control group | 71.2 ± 8.3 | 54% |
| KC3 strain alone | 89.9 ± 10.3 | 42% |
| Leonurus japonicus | 92.4 ± 9.8 | 41% |
| extract alone | | |
| Mixture of KC3 | 66.7 ± 7.1 | 57% |
| strain + Leonurus | | |
| japonicus extract | | |

As confirmed in Table 6 and FIGS. 4 and 5, it was confirmed that, compared to the bronchial damage-induced group by air pollutants, the total number of BAL cells confirmed in the BAL fluid was significantly reduced in the case of the treatment with the mixture of the KC3 strain and the Leonurus japonicus extract, thereby confirming anti-inflammatory activity on the bronchial inflammation. In particular, it was confirmed that the anti-inflammatory activity was similar to that of the positive control group treated with dexamethasone that is used as an anti-inflammatory agent, compared to the groups to which only the KC3 strain was administered or only the Leonurus japonicus extract was administered alone, respectively, thereby confirming a synergic effect on the defense against respiratory damage caused by respiratory inflammation due to fine dust.

### Experimental Example 1-3

### Confirmation of Ratio of Number of Neutrophils to Total Number of Cells in BAL Fluid

In the case of treatment with the Lactobacillus plantarum KC3 strain, the Leonurus japonicus extract, or the composition including a mixture of the KC3 strain and the Leonurus japonicus extract, an experiment to confirm whether neutrophils exhibited an effect on the number of cells among the total number of cells in the BAL fluid.

For details, the methods and the positive control group were set in the same manner as in Experimental Example 1-3, and the experiment was carried out under the same conditions as described above. From the recovered BAL fluid, inflammatory immune cells, neutrophils, were stained by Diff-Qick staining, and a ratio of stained neutrophil cells to the total cells was confirmed, and the results are shown in Table 7.

TABLE 7
Confirmation of ratio of number of neutrophils
to total number of cells in BAL fluid

| | Ratio of number of | Inhibition rate |
|---|---|---|
| | neutrophils to total number | (based on |
| Division | of BAL cells (400X) | induced group) |
| Normal control | 1.0 ± 0.1 | |
| group | | |
| Induced group | 70.7 ± 10.3 | |
| Positive control | 35.5 ± 6.1 | 50% |
| group | | |
| KC3 lactic acid | 34.1 ± 4.2 | 52% |
| bacteria alone | | |
| Leonurus japonicus | 40.8 ± 5.2 | 42% |
| extract alone | | |
| Mixture of KC3 | 28.5 ± 4.1 | 60% |
| strains + Leonurus | | |
| japonicus extract | | |

As shown in Table 7, it was confirmed that the number of neutrophils increased by air pollutants such as fine dust was about 70.7±10.3 in the inflammation-induced group compared to the normal group, indicating that the number of neutrophils increased about 70% or more compared to the normal group. However, when the mixture of the KC3 strain and Leonurus japonicus extract was administered, the inhibitory rate based on the induced group was about 60%, confirming a significant effect of inhibiting respiratory inflammation to a significant degree compared to the groups administered with each component alone. That is, the inhibitory activity of the induced group on respiratory inflammation was confirmed to be significantly excellent compared to the inhibitory activity of the positive control group. Overall, it was confirmed that the group administered with the mixture of the KC3 strain and the Leonurus japonicus extract showed a synergic defense effect against damage caused by respiratory inflammation compared to the groups administered with each component alone.

### Experimental Example 1-4

### Confirmation of Inhibition of Expression of Inflammation Factors in BAL Fluid

An experiment was carried out to confirm whether the expression level of inflammation factors in the BAL fluid can be lowered, when treated with the Lactobacillus plantarum KC3 strain, the Leonurus japonicus extract, or the mixture thereof.

The experiment was carried out in the same manner as in Experimental Example 1-3, except that the number of cells in BAL fluid was measured. Instead of measuring the number of cells, ELISA was performed to measure the expression level of respiratory inflammation factors, such as IL-17A, TNF-α, and CXCL-1, expressed in BAL fluid. In detail, IL-17A antibodies (M1700, R&D Systems, Minneapolis, USA), TNF-α antibodies (MTA00B, R&D Systems, Minneapolis, USA), and CXCL-1 antibodies (MKCOOB, R&D Systems, Minneapolis, USA) were diluted with a buffer solution, coated microwells, and then cultured at 4° C. for 16 hours. Each well was washed with a buffer solution three times, and 10-fold diluted serum was dispensed at 100 μl per well. After being left at room temperature for 1 hour, the wells were washed twice. Then, 100 μl of Avidin-HRP-conjugated antibodies (DY007, R&D Systems, Minneapolis, USA) was treated thereon, and the wells were left again at room temperature for 1 hour, followed by washing again.

Tetramethylbenzidine (TMB) base solution (DY007, R&D Systems, Minneapolis, USA) was dispensed at 100 μl per well, and the well plate was left in the dark for 30 minutes. After 50 μl of a stopping solution (DY007, R&D Systems, Minneapolis, USA) was treated thereon, absorbance of the cells was measured at 450 nm. By the ELISA, the expression levels of IL-17A, TNF-α, and CXCL-1 were determined, and the results are shown in Table 8.

TABLE 8
Confirmation of expression levels of inflammation factors in BAL fluid

| | Concentration (pg/mL) (inhibition rate (%) based on induced | | | | | |
|---|---|---|---|---|---|---|
| | group is indicated in parentheses) | | | | | |
| Division | IL-17A | | TNF-α | | CXCL-1 | |
| Normal control | 15.3 ± 3.2 | | 52.4 ± 7.7 | | 55 ± 6.1 | |
| group | | | | | | |
| Induced group | 33.5 ± 5.7 | | 120.1 ± 13.4 | | 201 ± 13.7 | |
| Positive control | 19.7 ± 6.3 | (41%) | 64.5 ± 7.7 | (46%) | 122 ± 12.4 | (39%) |
| group | | | | | | |
| KC3 strain alone | 23.3 ± 1.7 | (30%) | 73.4 ± 8.8 | (39%) | 141 ± 11.1 | (30%) |
| Leonurus japonicus | 19.8 ± 3.3 | (41%) | 69.3 ± 7.8 | (42%) | 110 ± 10.7 | (45%) |
| extract alone | | | | | | |
| Mixture of KC3 | 14.8 ± 5.3 | (56%) | 50.3 ± 6.1 | (58%) | 85.9 ± 9.2 | (5 7%) |
| strain + Leonurus | | | | | | |
| japonicus extract | | | | | | |

As shown in Table 8, it was confirmed that, as a result of measuring the amounts of inflammation biomarkers, such as IL-17A, TNF-α, CXCL-1, and the like, in BAL fluid, the levels of the inflammation biomarkers (e.g., IL-17A, TNF-α, and CXCL-1) having increased respiratory damage by air pollutants were significantly reduced by the treatment with the mixture of the KC3 strain and the Leonurus japonicus extract by about 56% or more in all three inflammation biomarkers. When even compared with the groups treated with each component alone, such reduction is regarded as a synergic defense effect on respiratory damage through respiratory inflammation defense to a significant degree. In addition, it was confirmed that the mixture exhibited significantly superior inhibitory activity on bronchial inflammation compared to the positive control group.

Experimental Example 2. Measurement of chronic obstructive pulmonary disease (COPD) biomarkers in blood

When treating the composition with the Lactobacillus plantarum KC3 strain, the Leonurus japonicus extract, or the mixture thereof, an experiment was carried to confirm whether the composition exhibited a therapeutic effect on COPD which is a type of respiratory diseases that can induced by fine dust.

In detail, an evaluation test was performed using ELISA to measure symmetric dimethylarginine (SDMA), which is a COPD biomarker, in the blood.

Serum was isolated from the blood collected from the heart of the BALB/c male mouse (7-week-old, 20 g to 25 g, Orient Bio) of Experimental Example 1, and SDMA antibodies (MBS2605912, MyBioSource, SanDiego, Calif., USA) were diluted with a buffer solution and coated microwells (96 wells, SPL 30096, Allforab), and then cultured at 4° C. for 16 hours. Each well was washed with a buffer solution three times, and 10-fold diluted serum was dispensed at 100 μl per well.

After being left at room temperature for 1 hour, the wells were washed twice. Then, 100 μl of Avidin-HRP-conjugated antibodies (DY007, R&D Systems, Minneapolis, USA) was treated thereon, and the wells were left again at room temperature for 1 hour, followed by washing again. A TMB base solution (DY007, R&D Systems, Minneapolis, USA) was dispensed at 100 μl per well, and the well plate was left in the dark for 30 minutes. After 50 μl of a stopping solution (DY007, R&D Systems, Minneapolis, USA) was treated thereon, absorbance of the cells was measured at 450 nm. After treating the composition with each of the Lactobacillus plantarum KC3 strain, the Leonurus japonicus extract, the mixture thereof, results of measuring the amount of SDMA, which is a COPD biomarker, in the blood are shown in Table 9.

TABLE 9
Measurement of SDMA as COPD biomarker in blood

| | SDMA level (μg/mL) | Inhibition rate (based on |
|---|---|---|
| Division | in serum | induced group) |
| Normal control | 7.1 ± 3.1 | |
| group | | |
| Induced group | 17.2 ± 3.7 | |
| Positive control | 10.1 ± 1.9 | 41% |
| group | | |
| KC3 strain alone | 12.1 ± 3.3 | 30% |
| Leonurus Japonicus | 7.2 ± 1.7 | 58% |
| extract alone | | |
| Mixture of KC3 | 4.1 ± 1.3 | 76% |
| strain + Leonurus | | |
| japonicus extract | | |

As shown in Table 9, it was confirmed that, as previously confirmed in the induced group, the level of SDMA which is a COPD biomarker increased by air pollutants reduced by the administration of the mixture of the KC3 strain and the Leonurus japonicus extract, thereby confirming the inhibitory activity on COPD by about 76% or more compared to the induced group. Overall, it was confirmed that the effect of the administration of the mixture as described above was significantly excellent even compared to the administration of each component alone, thereby confirming a synergic effect of the mixture also for the treatment or prevention of a respiratory disease, so as to inhibit COPD which is a representative respiratory disease.

Formulation Examples of the composition including the mixture of the Lactobacillus plantarum KC3 strain and the Leonurus japonicus extract according to an aspect are described below, but the present disclosure is not intended to be limited thereto, but only to be described in detail.

### Formulation Example 1

### Preparation of Powder

| | |
|---|---|
| Mixture of Lactobacillus plantarum KC3 | 20 mg |
| strain and Leonurus japonicus extract | |
| Lactose | 100 mg |
| Talc | 10 mg |

The components above were mixed and filled in an airtight bag to prepare a powder.

### Formulation Example 2

### Preparation of Tablet

| | | |
|---|---|---|
| Mixture of Lactobacillus plantarum KC3 | 10 | mg |
| strain and Leonurus japonicus extract | | |
| Corn starch | 100 | mg |
| Lactose | 100 | mg |
| Magnesium stearate | 2 | mg |

The tablet was prepared by mixing the above components and en-tableting the same, according to an existing tablet formation method.

### Formulation Example 3

### Preparation of Capsule

| | | |
|---|---|---|
| Mixture of Lactobacillus plantarum KC3 | 10 | mg |
| strain and Leonurus japonicus extract | | |
| Crystalline cellulose | 3 | mg |
| Lactose | 14.8 | mg |
| Magnesium stearate | 0.2 | mg |

A capsule was prepared by mixing the components above and filling a gelatin capsule with the mixture, according to an existing capsule formation method.

### Formulation Example 4

### Preparation of Injection

| | | |
|---|---|---|
| Mixture of Lactobacillus plantarum KC3 | 10 | mg |
| strain and Leonurus japonicus extract | | |
| Mannitol | 180 | mg |
| Distilled water for injection | 2974 | mg |
| Na₂HPO₄12H₂O | 26 | mg |

According to an existing injection formation method, an injection was prepared based on the component contents above per 1 ampoule (2).

### Formulation Example 5

### Formation of Liquid

| | | |
|---|---|---|
| Mixture of Lactobacillus plantarum KC3 | 10 | mg |
| strain and Leonurus japonicus extract | | |
| Isomerized glucose syrup | 10 | g |
| Mannitol | 5 | g |

| | |
|---|---|
| Purified water | optimum amount |

According to an existing preparation method for a liquid formulation, each component was added to purified water and dissolved therein, and an optimum amount of lemon flavor was added and mixed with the components above. Then, purified water was added thereto so that the total volume was adjusted to 100 ml, and the resultant solution filled in a brown bottle and sterilized, thereby preparing a liquid formulation.

### Formulation Example 6

### Preparation of Health Food

| Mixture of Lactobacillus plantarum KC3 | 1,000 | mg |
|---|---|---|
| strain and Leonurus japonicus extract | | |
| Vitamin mixture | optimum amount | |
| Vitamin A acetate | 70 | ug |
| Vitamin E | 1.0 | mg |
| Vitamin B1 | 0.13 | mg |
| Vitamin B2 | 0.15 | mg |
| Vitamin B6 | 0.5 | mg |
| Vitamin B12 | 0.2 | ug |
| Vitamin C | 10 | mg |
| Biotin | 10 | ug |
| Nicotin acid amide | 17 | mg |
| Folic acid | 50 | ug |
| Calcium pantothenate | 0.5 | mg |
| Minearl mixture | optimum amount | |
| Ferrous sulfate | 1.75 | mg |
| Zinc oxide | 0.82 | mg |
| Magnesium carbonate | 25.3 | mg |
| Monopotassium phosphate | 15 | mg |
| Dipotassium phosphate | 55 | mg |
| Potassium citrate | 90 | mg |
| Calcium carbonate | 100 | mg |
| Magnesium chloride | 24.8 | mg |

The compositional ratios of the vitamins and minerals in the mixture were set based on components relatively suitable for health food in preferable Examples, but the mixing ratios may be arbitrarily modified. According to the health food preparation methods in the art, each component may be mixed to prepare granules which will be then used for the preparation of the health food composition.

### Formation Example 7

### Preparation of Health Beverage

| | | |
|---|---|---|
| Mixture of Lactobacillus plantarum KC3 | 1,000 | mg |
| strain and Leonurus japonicus extract | | |
| Citric acid | 1,000 | mg |
| Oligosaccharide | 100 | g |
| Pulm concentrate | 2 | g |
| Taurin | 1 | g |

| | |
|---|---|
| By addition of purified water | 900 ml in total |

According to an existing method of preparing a health beverage, the components above were mixed and heated at 85° C. for about one hour while stirring. The prepared solution was filtered and collected in a sterilized 2 L container. The container was sealed, sterilized, and stored in a refrigerator to be used for the preparation of a health beverage composition.

1. A pharmaceutical composition for preventing or treating a respiratory disease, the pharmaceutical composition comprising, as active ingredients, one or more selected from the group consisting of a Lactobacillus plantarum KC3 strain (Accession No: KCTC13375BP), a culture thereof, a fermentation solution thereof, a lysis solution thereof, an extract thereof, and a concentrate of the culture and a Leonurus japonicus extract.

2. The pharmaceutical composition of claim 1, wherein the one or more selected from the group consisting of the Lactobacillus plantarum KC3 strain (Accession No: KCTC13375BP), the culture thereof, the fermentation solution thereof, the lysis solution thereof, the extract thereof, and the concentrate of the culture and the Leonurus japonicus extract are included at a weight ratio in a range of 1 to 0.1 to 10 (w/w).

3. The pharmaceutical composition of claim 1, wherein the Lactobacillus plantarum KC3 strain is included at a density in a range of 0.1 x 10⁹ CFU/cell to 1.0×10⁹ CFU/cell.

4. The pharmaceutical composition of claim 1, wherein the Leonurus japonicus extract is extracted from water, a C₁-C₁₀ alcohol, or a mixture thereof.

5. The pharmaceutical composition of claim 1, wherein the respiratory disease is any one of respiratory diseases selected from the group consisting of bronchitis, tuberculosis, chronic pulmonary disease, rhinitis, otitis media, viral respiratory disease, sore throat, tonsilitis, pneumonia, asthma, and chronic obstructive pulmonary disease (COPD).

6. The pharmaceutical composition of claim 1, wherein the pharmaceutical composition inhibits expression of IL-17A, TNF-α, and CXCL-1.

7. Health functional food for preventing or ameliorating a respiratory disease, the health functional food comprising, as active ingredients, one or more selected from the group consisting of a Lactobacillus plantarum KC3 strain (Accession No: KCTC13375BP), a culture thereof, a fermentation solution thereof, a lysis solution thereof, an extract thereof, and a concentrate of the culture and a Leonurus japonicus extract.

8. A food composition for preventing or ameliorating a respiratory disease, the food composition comprising, as active ingredients, one or more selected from the group consisting of a Lactobacillus plantarum KC3 strain (Accession No: KCTC13375BP), a culture thereof, a fermentation solution thereof, a lysis solution thereof, an extract thereof, and a concentrate of the culture and a Leonurus japonicus extract.

9. A quasi-drug composition for preventing or ameliorating a respiratory disease, the quasi-drug composition comprising, as active ingredients, one or more selected from the group consisting of a Lactobacillus plantarum KC3 strain (Accession No: KCTC13375BP), a culture thereof, a fermentation solution thereof, a lysis solution thereof, an extract thereof, and a concentrate of the culture and a Leonurus japonicus extract.

10. A method of preventing or treating a respiratory disease, the method comprising administering a composition to a subject in need thereof, the composition comprising, as active ingredients, one or more selected from the group consisting of a Lactobacillus plantarum KC3 strain (Accession No: KCTC13375BP), a culture thereof, a fermentation solution thereof, a lysis solution thereof, an extract thereof, and a concentrate of the culture and Leonurus japonicus extract.


SEQUENCE LISTING (1 sequence)

SEQ ID NO: 1 - DNA, 1509 bp, Artificial
  16s rRNA of Lactobacillus plantarum KC3 (KCTC13375BP)
tatggctcaggacgaacgctggcggcgtgcctaatacatgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagtttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagtcgtaca